(19) United States
(12) Patent Application Publication
(10) Pub. No.: US 2019/0105461 A1
(43) Pub. Date: Apr. 11, 2019

(54) MELANOPSIN STIMULATION USING NEURALLY PRIMED CONTENT

(71) Applicant: Ido Banai, Mae Hong Son (TH)

(72) Inventor: Ido Banai, Mae Hong Son (TH)

(21) Appl. No.: 16/151,831

(22) Filed: Oct. 4, 2018

Related U.S. Application Data
(60) Provisional application No. 62/568,972, filed on Oct. 6, 2017.

(51) Int. Cl. A61M 21/02; H04N 11/20; H04N 9/77

(52) U.S. Cl. CPC A61M 21/02; A61M 2021/005; H04N 9/77; H04N 11/20

(57) ABSTRACT

Systems and methods are disclosed for creating high dynamic range (HDR) Neurally Primed Content (NPC) for stimulating a content viewer's melanopsin vision system (MVS), based on standard content, for viewing on a high dynamic range (HDR) display. The color and brightness information of a piece of standard content are analyzed. Based on the analysis, a color lookup table (CLUT) is created for use in converting the standard content color and brightness information to a higher bit depth representation of the color and brightness information. Using the CLUT and the standard content, a high dynamic range (HDR) logical representation of the content is creating having the higher bit depth. Finally, an NPC physical color representation of the higher bit depth HDR logical representation of the content is created for viewing on an HDR display.

### CROSS-REFERENCE TO RELATED APPLICATIONS

This application claims priority under 35 U.S.C. § 119(e) to U.S. Provisional Patent Application No. 62/568,972, filed Oct. 6, 2017, entitled “Melanopsin Stimulation Using Neurally Primed Content,” the disclosure of which is hereby incorporated by reference in its entirety.

### BACKGROUND

Computing has been advancing at a stellar pace and computing devices are now an inseparable part of our lives. Many of us spend the majority of our waking hours working indoors in front of some type of digital screen, for example, looking at our cellphone or sitting in front of a computer in a cubicle. We have created a new paradigm for humans to exist in—indoors, distinctly different from hundreds of thousands of years driving our evolution to sustain our lives outside, in nature, under the bright open sky.

For at least the foreseeable future, people will continue in this new digital age to spend the majority of our time indoors, our primary source of lighting being either a lighting fixture in the room or a computer screen we are sitting in front of. As we spend more and more time away from the natural world where we are outside in constant exposure to the sun and the sky, we start to suffer from natural deficiencies of ambient light qualities which regulate certain basic human biological functions.

One of these deficiencies has its roots in the lack of exposure to naturally occurring blue sky light, which wreaks havoc on a person's circadian rhythm and associated sense of energy and vitality. These deficiencies can result in some very specific: disorders. A prime example is season effective disorder (SAD), which occurs primarily in geographic areas that are exposed to very little sunlight during winter months greatly reducing daylight hours, such as winter in the Nordic states, Russia, Canada, and Alaska. So the lack of blue light is often diagnosed as the cause for extreme fatigue or irregular sleeping cycles when daylight is limited. These symptoms are becoming more and more prevalent as growing numbers of people all over the world spend increasing amounts of time indoors and away from natural blue light.

Another example of disruption in the pattern of natural sunlight is so-called jet lag. While not caused by the lack of blue light, this disruption under stimulates the biological system in the same way a blue light deficiency would. After quickly traveling across many time zones, a person's circadian rhythm may be disrupted because the sun is up when the body expects it to be night. Consequently, the body wants to sleep during the day even though it is not the correct time to sleep.

### BRIEF SUMMARY OF THE INVENTION

System and method for creating high dynamic range (HDR) Neurally Primed Content (NPC) for stimulating a content viewer's melanopsin vision system (MVS), based on standard content, for viewing on a high dynamic range (HDR) display. The color and brightness information of a piece of standard content are analyzed. Based on the analysis, a color lookup table (CLUT) is created for use in converting the standard content color and brightness information to a higher bit depth representation of the color and brightness information. Using the CLUT and the standard content, a high dynamic range (HDR) logical representation of the content is creating having the higher bit depth. Finally, an NPC physical color representation of the higher bit depth HDR logical representation of the content is created for viewing on an HDR display.

By utilizing the encoding of naturally occurring Blue light onto a new format of digital content which allows for the representation higher dynamic range brightness values, which, in turn, when displayed on a new type of display systems which are capable of displaying this new type of information will create images that are not only perceptually brighter but actually be able to provide a meaningful reproduction of real world ambient lightening conditions to allow the viewer to influence the circadian rhythm and promote a sense of energy and well-being correct circadian sleeping rhythm disorders and reduce depression just through the normal viewing of content indoors.

Currently, as the relationship between the circadian rhythm and the Melanospin Vision System have only recently been established, scientist and doctors are only now starting to better understand the Melanospin Vision System which guides the circadian rhythm hence have only been able to treat a very specific range of extreme conditions such as seasonal effective disorder and not much else.

Traditionally this has only been done in the way of using hardware light devices which subject the patient to the specific required value of light by the use of bright LED requiring the patient to sit or lay down for an extended period of time in front of a specific piece of hardware which produces similar to real world lighting conditions. However, this requires specific hardware, diagnosis and often a cumbersome process for the patient to receive the light as medicine.

By the use of a new type of content on new much brighter display systems such as HDR TV's and mobile devices we can effectively stimulate the melanospin vision system and effect the circadian rhythm allowing a viewer to gain energy and vitality, or correct a sleeping disorder simply by viewing digital content which has been encoded in a specific way with an HDR display system. This now can be done effectively on television, computer, VR headsets and mobile devices. Allowing both targeted treatment for specific conditions as well as supplemental use to increase energy and vitality while indoors for extended periods of time.

Neurally Primed Content is a new type of content which leverages on several biological mechanisms and methodologies to create content that when combined stimulates biological and neurological responses from the viewer.

It uses a range of stimuli from targeted light exposure at specific wavelengths and frequencies at high brightness values to nonrealistic depth augmentation all embedded into standard digital content. Ranging from images, video, film, and light field volumetric datasets to fully animated CG content such as games, virtual reality scenes and augmented reality projections. When this content is presented through a display system bright enough, the coded brightness value will stimulate various biological responses from the viewer via (but not limited to) stimulation of the melanospin vision system.

Some aspects of stereoscopic NPC can be furthered adjusted and fine-tuned through the use of closed neural feedback loop systems with EEG readers or other BMI devices

NPC will also make use of Bi-Neural sound elements, the syncing of stereoscopic augmentation effects to audio elements.

A novel method to stimulate the melanopsin vision system to affect a host of biological mechanisms via the intrinsically photosensitive retina! ganglion cells(ipRGCs) through specific targeted light exposure from digital content (image,Video) or software solutions (VR/AR/Mobile app) called Neurally Primed Content (NPC) on capable high dynamic range (HDR) display systems with increased brightness capabilities (e.g., HDR-10, Dolby vision, Rec 2020 or higher color space).

NPC is to be displayed on an electronic screen, or a projected surface, or a light field volumetric display system with the explicit purposes of increasing a subject's energy levels and wakefulness via stimulation of the melanopsin vision system including underexposed ipRGCs, thereby effecting changes in circadian rhythm and stimulating an endogenous release of DMT (N,N-Dimethyltryptamine) within the brain.

A digital light source at a specific narrow or specific wavelength of light residing somewhere around 479 nm is displayed through a bright capable display system (e.g., an HDR display) for digital devices which is perceived as a bright white light but is actually naturally occurring blue skylight at a very high brightness intensity for the purposes of stimulating the ipRGCs at a peak response wavelength for stimulation of various biological mechanisms.

The use of HDR video technology and displays that are brighter than existing screens is a key enabling factor for the effectiveness of this process and such forth requires a different way of coding digital information in order to enable effective use.

Via the creation of digital content at a higher bit depth then 8 bit (e.g., 10 bit 12 bit, 14 bit 16 bit and 32 bit) of a light source of 479 nm wavelength set to value higher then 1 we will represent a color which is visibly white due to its brightness but actually at a specific desired peak response wavelength to effect the ipRGC's.

Currently this can be done via the use of several proprietary methods:
Creation of a specific software package to generate the environment and light source as a mobile app, VR experience or computer software.
Generation of NPC or converting regular digital content to NPC via a proprietary VFX process producing a new NPC master.
Real time conversion of regular digital content via the use of a 3D LUT (look up table) on capable display systems, mobile devices and HMD's.

NPC also deploys nonrealistic dynamic depth augmentation for stereoscopic NPC. This involves several layers of depth augmentation based on 3 separate Z-depth augmentations. Base layer natural depth map generation, a Depth Script augmentation which is, contextually based animated augmentation and a third underlying layer of depth augmentation via the use of mathematical form constants overlaid over distinct planer surfaces. The second and third layers of depth augmentation can be effected by a closed neural loop system such as the use of a low latency EEG sensor system.

A novel method to convert existing digital content to Neurally Primed Content for e.g., entertainment, wellness and medical purposes. First, all digital content is converted to a higher then 8 Bit color bit depth, typically 32 Bit float in a complementary color space for HDR Video (e.g., HDR-10, Dolby vision, Rec.2020 color space or higher).

By using a method of matte extraction for the desired areas of the images we create a luminance matte that is used as a guide to replace the existing bright color values with corresponding lightness values which are higher then 1 with a specific color value guided by our choice color wavelength. Once the area of the image has been identified and the specific color wavelength selected the user determines the magnitude of the lightness desired remasters the content as NPC as a new rendered file. For real time display conversion a lookup table can be applied to the digital content in real time. This lookup table can be performed by a capable display system as a non-destructive overlay to any piece of content provided.

Through the use of a DCC (digital content creation) application such as Maya or Nuke an artist either creates new CG content or modifies existing digital imagery/video in order to embed higher lightness values in certain areas or object in the scene. These files then get written out as a HDR compatible file format or embed in a special mobile app/player to allow for proper playback and display optimization per device.

### Digital Video/Images

For digital images or video a user will employ a VFX application such as Nuke or After effects to create a matte of the desired area to effect this matte can be created by a luminance based matte extraction, Rotoscopy, Chroma keying or any other method of matte extraction.

After a matte has been created a color correction will be applied to the footage in color bit-depth higher then 8 bit allowing to generate new color values with values higher then 1. These color values will not actually be white but a specific pre-determined color value or range based on the corresponding desired color wavelength that will generate a peak ipRGC response. The method of color correction or augmentation are vast and varied and are dependent on the contextual parameters of both the content and its intended use. In some cases a color correction operation may be applied in others a completely new element may be introduced via a composite operation. Regardless, by coding these new color values above 1, when the content is displayed via the use of HDR capable display systems they will be perceived as white light source but in actuality will output a different wavelength then the original white light wavelength.

The same process also applies when dealing with the color values of volumetric point cloud data such as light fields with the added benefit of much more granular control over the volumetric structure of the lighting effect, its frequency and its falloff.

### EXAMPLE 1

A digital movie is projected on a HDR capable display with Neurally Primed Content generated on the fly by the display itself .The display is able to measure of all the bright pixels over a certain range and replaces them with the appropriate super peak frequency skylight color values in those areas, creating in real-time Neurally Primed Content out of any piece of content provided.

### CG NPC

When dealing with fully CG NPC the creation package of choice already offers tools to create both GPU driven real time scenes or rendered images and light fields which can be coded with these light values straight from the light source and shaders within the host application.

### EXAMPLE 2

A computer generated scene of a beach where the viewer is staring directly at the sun. Both the sky and the sun have been specifically coded with peak light response frequencies around 479 nm, which is the peak frequency to stimulating the Melonospin vision system. By simply staring into the sun on an HDR capable display the user is able to stimulate their biological Melonospin vision system and gain a host of medical and wellness benefits.

### EXAMPLE 3

A mobile VR mobile app where users use their HDR-10 Capable mobile device with a mobile VR head-set solution when activated the app allows them to experience a sustained targeted sequences of peak frequency skylight in order to treat various sleeping disorders and medical conditions and improve wakefulness.

### Unclouded App

By using a mobile cell phone device with an HDR-10 capable display we are able to code these higher light values into existing digital content or specifically generated NPC and NPC mobile applications for mobile phones. Neurally Primed Content allows us to turn the mobile device into a light therapy device to stimulate the Melanopsin vision system with the use of specific wavelengths of light presented on the screen through specifically converted digital content or an actual application on the phone. The viewer will wear a headset for their mobile device and use it as a VR HMD for the duration of the experience.

Unclouded works with any HDR-10 capable mobile device, it can be used with any mobile VR headset solution, from cardboard to google daydream and Samsung VR. Depending on the type of experience the patient chooses it will last 5-30 minutes. A patient will be seated, laying down or in a meditation pose with headphones, they will be transported to a surreal natural environment meant to calm them down and allow them to fall into a more meditative state.

With the use of basic color cognitive science, Bi-neural audio and a voice over we will guide the patient into a state in which they are more prone to close their eyes. Once their eyes are closed the actual biological stimulation of the Melanopsin vision system can be done safely.

A natural light source (typically the sun) within the VR environment will become brighter and fill the screen, in this process the wavelength (color) of the light source will change to the specific wavelength which allows for peak response by the IPRGC's thus stimulating the circadian rhythm via the Melanopsin vision system and promoting a state of energy, wakefulness and well-being.

### Explanation of Attached Road Map

The road map below lists the core medical and wellness application separated by each biological mechanism that is effected by Melanospin stimulation and some of its possible uses. Currently the focus is on the use of Neurally Primed Content for medical and wellness applications.

In accordance with another exemplary aspect, the present invention provides a system and apparatus for stimulation of the Melanopsin vision system comprising: Melanopsin stimulant content; means for converting standard content to Melanopsin stimulant content; and an apparatus configured to deliver the Melanopsin stimulant content to a user.

The apparatus for delivering the Melanopsin stimulant content comprises an HDR enabled display, an array of at least one light emitting diode, or head mounted display system. The Melanopsin stimulant content is digital content encoded with specific visible and non-visible frequencies of light.

The exemplary embodiments of the present invention also provide a method for creating Melanopsin stimulant content by converting standard content comprising: analyzing a color and brightness content of a piece of standard content; based on the analysis, developing a color lookup table (CLUT) for use in converting the standard content color and brightness content to a higher bit depth representation of the color and brightness content; using the CLUT and the standard content, creating a Melanopsin stimulant content having high dynamic range (HDR) logical representation of the content having the higher bit depth; and creating a Melanopsin stimulant content physical color representation of the higher bit depth HDR logical representation of the content for viewing on an HDR display.

The step of creating the Melanopsin stimulant content comprises encoding alternative specific light frequencies in visible light values of at least one generated digital representation, including an image, video, or plenoptic data set that will stimulate intrinsically photosensitive retinal ganglion cells (IPRGC) and the melanopsin vision system to generate a host of biological responses. The step of creating the Melanopsin stimulant content can be conducted on a server computer operatively coupled via a network to a user computing device having an HDR display. The step of creating the Melanopsin stimulant content can be conducted on a user computing device having an HDR display.

In accordance with another exemplary aspect, the prevent invention provides a system for creating high dynamic range (HDR) Melanopsin stimulant content based on standard content content, for viewing on a high dynamic range (HDR) display, comprising: a standard content analysis component; a lookup table component; a high dynamic range (HDR) logical content creation component; and a Melanopsin stimulant content physical color creation component.

The foregoing exemplary embodiments provides a system and method for creating Melanopsin stimulant content intended to stimulate a host of biological responses in a viewer via the viewing of digital content that has been encoded with specifies visible and non-visible frequencies of light on a wide range of capable display systems. These responses range from an increased sense of energy, vitality and wakefulness via stimulation of the melanopsin vision system to regulate and support a healthy circadian rhythm even through extreme periods of time spent in front of a digital display system or in situations where exposure to the sky light is not possible. Secondly targeted pulsations, frequencies at greater intensities can promote an endogenous release of DMT as increased stimulation of the IPRGC is able to trigger responses further down the path of the melanopsin vision system as it links to other mechanisms in the brain.

Furthermore, applications from just direct IPRGC stimulation with the same light frequencies can yield positive healing effects for retinal nerve fiber layer thinning in glaucoma patients. It is also possible to use NPC with the use of gene therapy to create new melanospin sensitive Opsins in the brain to regulate firing imbalances of excitable vs. resistant neurons in the brain (a key aspect of Autism disorder).

Other features and advantages of the subject disclosure will be apparent from the following more detailed description of the exemplary embodiments, taken in conjunction with the accompanying drawings which illustrate, by way of example, the principles of the subject disclosure.

### BRIEF DESCRIPTION OF THE SEVERAL VIEWS OF THE DRAWINGS

The foregoing summary and the following detailed description of certain exemplary embodiments and aspects will be better understood when read in conjunction with the accompanying drawings. The drawings illustrate exemplary embodiments and aspects of the disclosure. It should be understood that the invention is not limited to the precise arrangements and instrumentalities shown and described. Rather, the invention and its scope are defined by the appended claims and their equivalents.

In the drawings:

FIG. 1 is a block diagram illustrating physical components of an exemplary computing device with which aspects of the disclosure may be practiced;

FIGS. 2A and 2B are simplified diagrams of a mobile computing device with which aspects of the present disclosure may be practiced;

FIG. 3 is a simplified block diagram of a distributed computing system in which aspects of the present disclosure may be practiced;

FIGS. 4A and 4B illustrate aspects of abstracted exemplary look-up tables, similar in nature to color look-up tables (CLUTs) with which aspects of the disclosure may be practiced; and

FIG. 5 is a diagram of a system for modifying the output of NPC being viewed by a subject on a HDR device.

### DETAILED DESCRIPTION

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any matter not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the subject disclosure can be presented in a range format. It should be understood that the description in range format is merely for convenience and brevity and should not be construed as an inflexible limitation on the scope of the exemplary embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the exemplary embodiments can be practiced without one or more of the specific features or advantages of a particular embodiment. In other instances, additional features and advantages may be recognized in certain embodiments that may not be present in all exemplary embodiments of the subject disclosure.

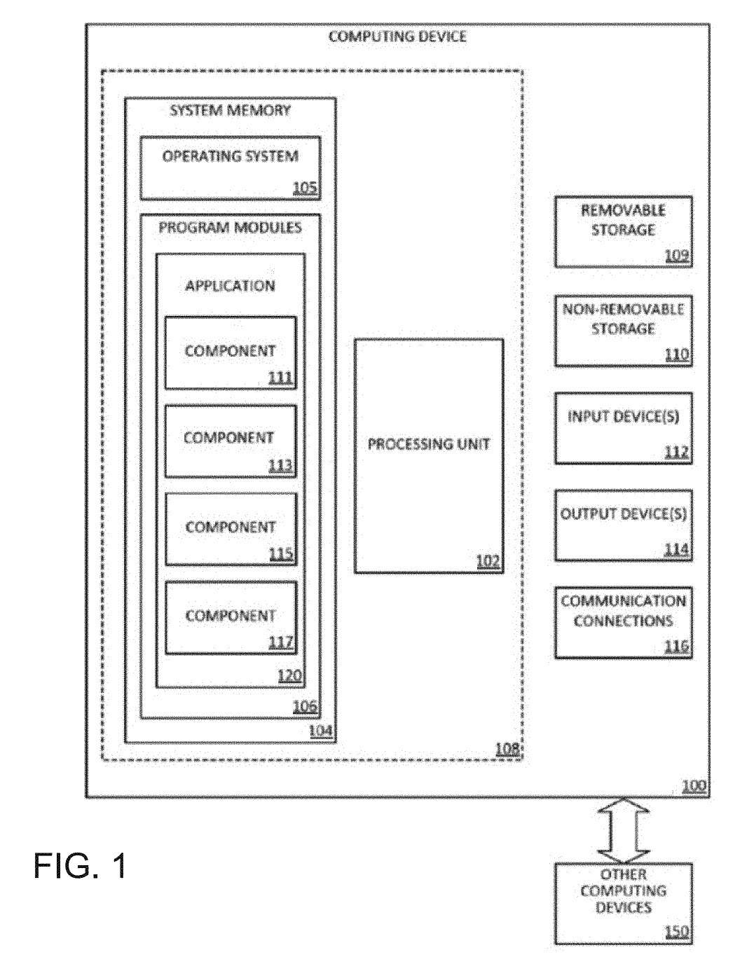

As an initial matter, FIGS. 1-3 illustrate exemplary operating environments in which aspects of the disclosed embodiments may be practiced. The devices and systems illustrated and described with respect to FIGS. 1-3 are presented for purposes of example and illustration, and are not limiting of other computing devices and configurations that may be utilized for practicing aspects of the described embodiments.

FIG. 1 is a block diagram illustrating physical components of a computing device 100 with which aspects of the disclosure may be practiced. The computing device 100 may have computer executable instructions for implementing aspects of an information retrieval and delivery system. In embodiments, computing device 100 can be illustrative of a mobile user device, or a server computing device, or both. In a basic configuration the computing device 100 may include at least one processing unit 102 and a system memory 104. Depending on the configuration and type of computing device, the system memory 104 may comprise, but is not limited to, volatile storage (e.g., random access memory), non-volatile storage (e.g., read-only memory), flash memory, or any combination of such memories. The system memory 104 may store an operating system 105 and one or more program modules 106 suitable for running application 120. The operating system 104, programs modules 106, and application 120 with one or more components shown as 111, 113, 115, and 117, all comprise computer executable instructions that can be executed to realize the methods disclosed herein.

It is contemplated that the operating system 105 running on a mobile user device may be a smart phone running either the iOS by Apple Computer, or the Android operation system by Google, although other mobile devices and mobile device operating systems may be used. Moreover, it is contemplated the server may be a rack-mounted server running a version of Windows by Microsoft, or any of a variety of Unix-based operating systems, although other server configurations and server operating systems may be used. However, disclosed embodiments may be practiced in connection with hardware configurations, operating systems, program modules, applications, and/or application components other than those illustrated and described, and are not limited to any particular application or system.

In general, the operating system 105 controls aspects of the operation of the computing device elements illustrated in FIG. 1 within dashed line 108. The computing device 100 may have additional features or functionality. For example, it may also include additional data storage devices (removable and/or non-removable) such as, for example, magnetic disks, optical disks, or magnetic tape. Such additional storage is illustrated in FIG. 1 by removable storage device 109 and non-removable storage device 110.

A plurality of program modules and data files may be stored in memory 104. While executing on the processing unit 102, program modules 106 may perform processes including, but not limited to, herein described aspects and embodiments. Computing device 100 may be, for example, a server computing device operatively coupled to a user mobile computing device via a network, in which various aspects of the disclosure may be performed on the server, or on the user device, or both. Application 120 may include, for example, a standard content analysis component 111, a lookup table component 113, a high dynamic range (HDR) logical content creation component 115, and a Neurally Primed Content (NPC) physical color creation component 117.

It is understood that described embodiments may be practiced using electrical circuits comprising discrete electronic elements, packaged or integrated electronic chips containing logic gates, a circuit utilizing a microprocessor, or on a single chip containing electronic elements or microprocessors. For example, embodiments may be practiced using an application specific integrated circuit (ASIC), or a system-on-a-chip (SOC), where each or many of the components illustrated in FIG. 1 may be integrated onto a single integrated circuit. Such an ASIC or SOC device may include one or more processing units, graphics units, communications units, system virtualization units and various application functionality, all of which may be integrated onto a substrate as a single integrated circuit. When operating via an ASIC or SOC, the functionality described herein may be operated via application-specific logic integrated with other components of the computing device 100, which may be included on the integrated circuit. Embodiments may also be practiced using other technologies capable of performing logical operations such as, for example, AND, OR, and NOR, including but not limited to mechanical, optical, and quantum technologies. Aspects of disclosed embodiments may also be practiced within a general purpose computer. In such case, the computer elements utilized together with the computer instructions running on them realize a special purpose computer specific to those described aspects and embodiments.

Computing device 100 may have one or more input device(s) 112 such as a keyboard, a mouse, a pen, a microphone, a touch screen, etc. One or more output device(s) 114 may also be included, such as a display, a speaker, a printer, etc. For NPC viewing, the display may be an HDR display. Computing device 100 also includes one or more communication connections 116 allowing communication with other computing devices 150. Examples of suitable communication connections 116 include, but are not limited to, circuitry such as cellular and/or wifi radio frequency (RF) transmitter, receiver, and/or transceiver circuitry; busses such as universal serial bus (USB), parallel, and/or serial ports; and/or other network interface technologies known in the art.

The term computer readable media as used herein means a tangible computer readable digital data storage device. Computer storage media may include any or all of volatile and nonvolatile media, removable and non-removable media, implemented in any method or associated tangible technology for storage of information, such as computer readable instructions, data structures, and program modules. The system memory 104, the removable storage device 109, and the non-removable storage device 110 are all examples of computer storage media. Computer storage media may include RAM, ROM, electrically erasable programmable read-only memory (EEPROM), flash memory or other memory technology, CD-ROM, DVD or other optical storage, magnetic tape cassette storage, magnetic disk storage or other magnetic storage devices, solid state memory such as solid state drives (SSDs) or USB thumb drives, or any other article of manufacture which can be used to store information and which can be accessed by the computing device 100. Such computer storage media may be part of or operatively coupled to the computing device 100. As used herein, computer storage media does not include a carrier wave or other propagated or modulated data signal.

In embodiments, the mobile device and the server are operatively coupled via a network such as the Internet that includes communication media such as a wired network or direct-wired connection, and wireless media and interfaces such as radio frequency (RF), infrared, and/or other wireless media.

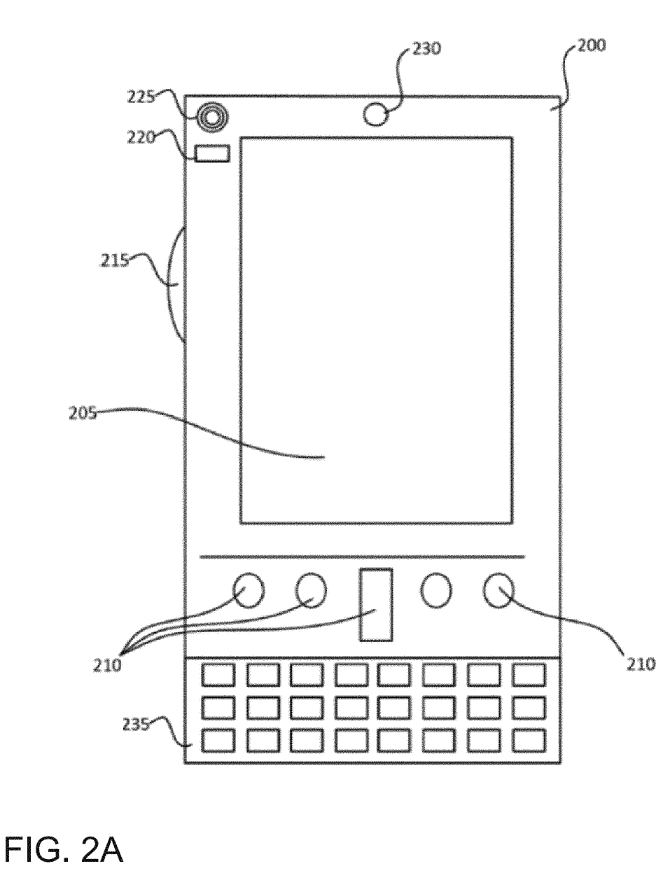

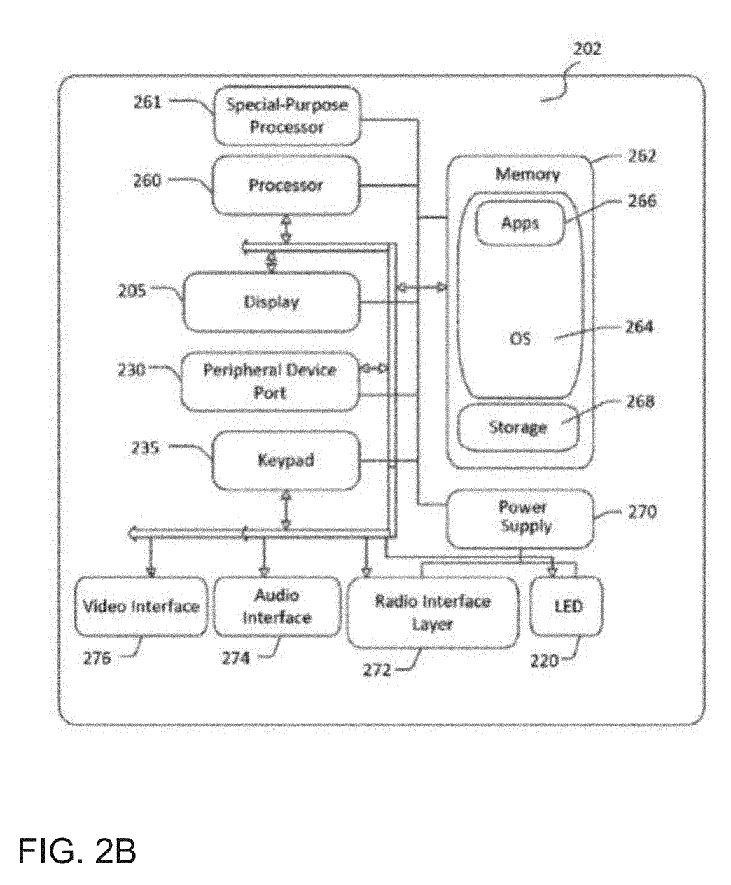

FIGS. 2A and 2B illustrate a mobile computing device 200, which may be referred to herein as a user device or a “mobile”. Mobile 200 may include some or all of the blocks shown in FIG. 1. In embodiments, mobile 200 may be a mobile telephone such as a smart phone as illustrated in FIGS. 2A and 2B, or a wearable computing device such as a smart watch, a tablet computer, a laptop computer, and the like, with which aspects of the disclosure may be practiced. In some aspects, such a mobile computing device may act as a client in a client-server arrangement. With reference to FIG. 2A in a basic configuration, the mobile computing device 200 is a smart phone or other handheld computer having both input elements and output elements. The mobile 200 may include one or more physical buttons 210 that act as user inputs to the mobile 200. Display 205 may also function as an input device (e.g., a touch screen display). A side input element 215 may also be included to allow further user input. For example, side input element 215 may be a rocker switch, a button, or any other type of physical input element. In various embodiments, mobile computing device 200 may incorporate more or fewer input elements. For example, a touchscreen embodiment may include a “soft” virtual keypad and/or other virtual inputs presented on the touchscreen. In other embodiments, the display 205 may not be a touch screen. Such embodiments may include a physical keypad 235. Output elements may include the display 205 showing a graphical user interface (GUI), a visual indicator 220 such as a light emitting diode (LED), and/or an audio transducer 225 such as a speaker. Mobile computing device 200 may also incorporate a vibration transducer for providing the user with tactile feedback. In yet another aspect, the mobile computing device 200 may include input and/or output ports, such as an audio input jack into which a microphone may be plugged, an audio output jack into which a headphone may be plugged, and a video output such as an HDMI port for sending or receiving video signals to or from an external video device.

FIG. 2B is a block diagram illustrating certain system aspects 202 within mobile computing device 200. One or more application programs 266 may be loaded into memory 262 and run under or in association with the operating system 264. Such application programs may include, for example, image capture and processing programs, word processors, spreadsheets, Internet browsers, messaging programs, and so forth. System aspects 202 also include a non-volatile storage area 268 within the memory 262. The non-volatile storage area 268 may be used to store persistent information that is not lost when user device 200 is powered down. The application programs 266 may use and store information in the nonvolatile storage area 268, such as information about personal contacts, text messages sent and received, and any information saved for later use by apps 266. For backup, a synchronization application may also reside within user device 200, programmed to interact with a host computer to store information at the host computer and to keep the stored information synchronized with the information stored in the non-volatile storage area 268. The host computer may be a server accessible via a network, or a directly connected personal computer (PC), for example.

The user device 200 includes a power supply 270, which may be implemented as an internal rechargeable battery. The power supply 270 may be coupled to an external power source such as an external battery, AC adapter, or powered docking cradle that supplements and/or recharges the internal battery.

User device 200 may also include a radio interface layer 272 that performs the function of transmitting and receiving radio frequency communications. The radio interface layer 272 facilitates wireless connectivity between user device 200 and a network operated by a communications carrier or service provider. Communications to and from the radio interface layer 272 are conducted under control of the operating system 264. As such, incoming communications received by the radio interface layer 272 may be directed by the operating system 264 to one or more application programs 266; and outgoing communications may be generated by application programs 266 and directed by the operating system 264 to the radio interface layer 272 and transmitted.

Visual indicator 220 may be used to provide visual notifications. Audio interface 274 may additionally or alternatively be used for producing audible notifications via the audio transducer 225. In the illustrated embodiment, the visual indicator 220 is a light emitting diode (LED) and the audio transducer 225 is a speaker. These devices may be directly coupled to the power supply 270 so that when activated, they remain on for a period of time controlled by the notification mechanism even though the processor 260 and other components might enter a low power state or shut down to conserve battery power. The LED may be programmed to remain on until the user takes an action related to the indicator. The audio interface 274 is used to provide audible signals to and receive audible signals from the user. For example, in addition to being coupled to the audio transducer 225, the audio interface 274 may also be coupled to a microphone to receive audible input, such as to facilitate a telephone conversation. In accordance with certain aspects and embodiments, the microphone may also serve as an audio sensor to facilitate control of notifications or to provide other user input. User device 200 may further include a video interface 276 that enables operation of an onboard camera 230 to record one or more still images, a video stream, and the like.

Mobile computing device 200 may have additional features or functionality. For example, the mobile 200 may also accommodate removable data storage devices such as a microSD card. Such additional storage is represented in FIG. 2B by the non-volatile storage area 268.

Information and data generated or captured by the mobile computing device 200 may be stored locally on the mobile 200, or may be stored on any number of external storage media. Such media may be accessed for example by the mobile 200 via the radio interface layer 272, or via a wired connection between the mobile 200 and a separate computing device associated with the mobile 200, such as an on-the-go (OTG) connected thumb drive for example. Alternatively or in addition, data may be sent to a server computer in a distributed computing network, such as the Internet, for storage. The data may thereafter be accessed by the mobile 200 via the radio interface layer 272 or other network interface to the server or other network storage location for retrieval. Similarly, such data/information may be readily transferred between computing devices and storage devices for use in accordance with the present disclosure.

Applications 266 may be stored in non-volatile storage 268, and loaded into memory 262 to run on processor 260 of mobile 200. A client app that operates in accordance with aspects of the present disclosure can be downloaded from an online program repository or “app store”. Other methods of a user device acquiring an app may alternatively be used. Such a program includes program instructions for implementing the user mobile device (or client) portion of a Neurally Primed Content (NPC) generation and display system as disclosed herein. In particular, as described in connection with FIG. 1, such an app may include a video analysis component 111, color space expansion component 113, color replacement component 115, and binaural generating component 117.

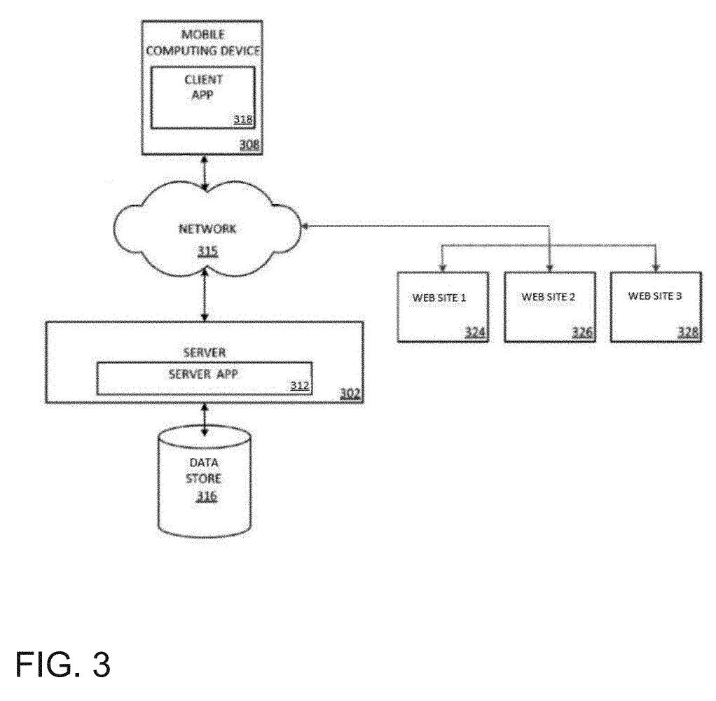

FIG. 3 illustrates further aspects of an exemplary embodiment of a system for generating and displaying NPC. As shown, the system may include a mobile computing device 308 running client app 318, and may also include a server computing device 302 running server app 312. Information and data for use by client app 318 and/or server app 312 may be stored in a tangible data storage device 316. Information stored in data store 316 may be accessed by server device 302 and by mobile 308. In embodiments, mobile 308 may access information stored in data store 316 directly via network 315, or indirectly by sending an appropriate message to server 302, which can then access the information identified in the message and send it to mobile 308. Mobile 308 can also access other resources via network 315, for example, one or more web servers. FIG. 3 illustrates a first web site 324 having a first web address, a second web site 326 having a second web address, and a third web site 328 having a third web address.

The foregoing generically described devices and systems are presented as non-limiting examples of environments in which embodiments and aspects of the methods, devices, and systems described herein may be practiced. In an exemplary aspect, the following disclosure will pertain to mitigating certain effects on the human body caused by changes in available sunlight in the environment. In particular, methods, devices, and systems to stimulate the melanopsin vision system (MVS) are described, using light sources that mimic certain aspects of natural sunlight. Melanopsin is a type of photopigment (unstable pigments that undergo a chemical change when they absorb light) belonging to a larger family of light-sensitive retinal proteins called opsins. In the human retina, there are additional opsins involved in the formation of visual images, including rhodopsin in the rod photoreceptor cells, and photopsin in the cone photoreceptor cells. Melanopsin is found in intrinsically photosensitive retinal ganglion cells (ipRGCs). The ipRGC are particularly sensitive to the absorption of short-wavelength (blue) visible light, and communicate directly to the area of the brain called the suprachiasmatic nucleus (SCN), also known as the central “body clock” in humans. Melanopsin thus plays an important non-image-forming role in the setting of circadian rhythms.

Surprisingly, the MVS has been found to be responsive to stimulation using display devices such as high dynamic range (HDR) computer monitors. In particular, HDR displays can present specially generated or modified imagery to stimulate the melanopsin vision system. Such content is referred to herein as Neurally Primed Content (NPC). Thereby, certain visible light-related conditions can be beneficially affected. For example, melanopsin stimulation may be used to treat conditions relating to circadian rhythm disruption, such as jet lag, shift work sleep disorder, advanced sleep phase disorder, delayed sleep phase disorder, irregular sleep-awake rhythm, and no-24 hour sleep-awake disorder. It may also be used to treat conditions at least partially attributable to melanopsin atrophy due to lack of natural skylight, such as seasonal affective disorder (SAD), spending excessive time indoors, and more particularly long term computer use, which may preclude a person's intake of natural sunlight, for example. In addition, there is evidence to suggest that viewing patterns of very bright flashing lights through closed eyelids (i.e., with eyes closed) may have a stimulating effect on the pineal gland, thereby promoting the release of natural DMT.

Producing NPC comprises encoding the color and intensity of a naturally occurring bright daylight sky (e.g., bright light blue) onto a new format of digital content. This allows for the representation of so-called high dynamic range (HDR) brightness values, which in turn, may be displayed on new types of HDR display systems capable of displaying such brightness values. This procedure results in generated images that are not only perceptually brighter than prior art display systems, but are actually able to provide a meaningful reproduction of real world outdoor ambient light conditions. Such light conditions can have a therapeutic effect. In particular, simulated daylight can be used to stimulate the melanopsin visual system, which in turn can mitigate seasonal depression and influence the circadian rhythm of the viewer, thereby promoting a sense of energy and well-being. Circadian sleeping rhythm disorders and light deficiency-induced depression can be mitigated, through the normal viewing of NPC-enhanced content. This can involve watching NPC-enhanced entertainment indoors (e.g., watching TV), viewing a computer screen having NPC-enhanced elements, and the like.

Currently, as the relationship between the circadian rhythm and the melanopsin vision system (MVS) have only recently been established and is currently being studied, scientists and doctors are only recently starting to better understand the MVS and its effect on circadian rhythm. Hence they have only been able to effectively treat a very narrow range of extreme conditions such as seasonal effective disorder.

The herein disclosed new type of digital can stimulate the viewer's MVS, especially when viewed on newly available, very bright digital display systems such as HDR TVs and mobile devices. In particular, this increases the degree of melanopsin stimulation experienced by the viewer, which can support and promote the viewer's energy and vitality. Alternatively or in addition, a viewer's circadian rhythm can be influenced in a controlled manner, allowing the viewer to overcome jet lag, or to correct certain sleep disorders. Such results may be obtained simply by viewing digital content which has been encoded in a specific way on an HDR display device. Such devices can include without limitation televisions, computer monitors, virtual reality (VR) head mounted displays (HMDs), and mobile devices. The specially encoded digital content can also be adapted to the particular HDR device on which it is played. Because the content brightness encoding, herein referred to as Neurally Primed Content (NPC) and the HDR output can both be controlled, the light values generated can be controlled as desired to produce bright light-induced neurologic stimulation effects. Such stimulation may, for example, be targeted as treatment of limited duration for light related medical disorders. It can also be used to supplement other light sources to increase energy and vitality while indoors for extended periods of time.

This Neurally Primed Content is a new type of content that takes advantage of several biological mechanisms and methodologies which, when combined, can stimulate biological and neurological responses from the viewer. NPC uses a range of stimuli based on generating light at controlled wavelengths, brightness, and duration. The light generated can be targeted to produce a desired light exposure by modifying generated wavelengths (i.e., colors) and brightness values (i.e., intensity) for desired lengths of time. Viewing content may be generated containing any desired degree of realistic to nonrealistic color depth augmentation, either entirely computer generated (CG), or based on and embedded into standard digital content. The content may include one or more images, video, film, and light field volumetric datasets, to fully animated computer generated (CG) content such as games, virtual reality (VR) scenes and augmented reality (AR) projections. When the content is presented on a display system that is bright enough to be effective, the coded brightness value will stimulate in a controlled manner various biological responses from the viewer, including but not limited to stimulation of the melanopsin vision system (MVS).

Some aspects of stereoscopic NPC may be further adjusted and fine-tuned through the use of a closed loop neural feedback system with one or more electroencephalograph (EEG) readers or other brain-machine interface (BMI) devices. NPC may also make use of binaural sound elements, and may synchronize visual stereoscopic augmentation effects to binaural audio elements (i.e., different left and right audio signals).

In an exemplary aspect, a novel method is used to stimulate the MVS to affect a variety of biological mechanisms via the intrinsically photosensitive retinal ganglion cells (ipRGCs). This may be achieved through specific targeted light exposure, which may be based on conventional visual digital content (images, video) and/or software generated solutions (VR/AR/games, etc.). NPC is most effective when presented on HDR display systems having increased brightness capabilities such as HDR10, Dolby Vision, Rec. 2020 color space, and the like.

Luminance is a photometric measure of the luminous intensity per unit area of light travelling in a given direction. In the context of display devices, it describes the amount of light emitted from a particular display area, in units of candela per square meter (cd/m²). A typical computer display may emit in the range of less than a hundred to a few hundred cd/m². Conventional standard content and high definition TV (HDTV) displays are similarly bright. But HDR displays emit in the range of 1,000-2,000 cd/m² or more, that is, on the order of 5-10 times as bright. In contrast, the sun high in the sky has a luminance of over a billion cd/m², and looking directly at it will quickly result in permanent blindness.

Gamma correction (or simply gamma) is a nonlinear operation used to encode and decode luminance values in video and still images. Gamma encoding of images and video optimizes the usage of bits when encoding an image by taking advantage of the non-linear manner in which humans perceive light and color. The perception of brightness under common illumination conditions (that is, not completely dark nor blindingly bright) follows an approximate power function, being more sensitive to relative differences between darker tones (e.g., details in shadows) than between lighter ones. This difference in light sensitivity is known as the Stevens power law for brightness perception. If images are not gamma-encoded, they allocate just as many bits to bright highlights that humans can't differentiate, as are allocated for shadow values that humans are much more sensitive to.

For purposes of viewing NPC, the increased luminance of an HDR display should be taken advantage of by commensurately increasing the luminance values of standard content encoded images and video. This increased luminance encoding for HDR requires a higher bit depth to encode than standard content. So pre-existing content produced for standard content must be converted into HDR video in which the full luminance capability of the HDR display can be used. This conversion can be accomplished either entirely by specially designed circuitry in hardware, such as a field programmable gate array (FPGA) or application-specific integrated circuit (ASIC), or such circuitry may be used in conjunction with a graphics processing unit (GPU) or a general purpose CPU. Alternatively or additionally, an appropriate software program running on a GPU or CPU may achieve the conversion of 8 bit standard content to higher bit HDR as NPC. Moreover, this conversion may take place on the fly as standard content-based NPC is being viewed, or it can be performed prior to viewing in high bit depth NPC. In addition to using the HDR display's full luminance to stimulate the MVS system, increasing the dynamic range of standard content can include increasing the general color brightness range, contrast ratio, and/or number of colors displayed on screen.

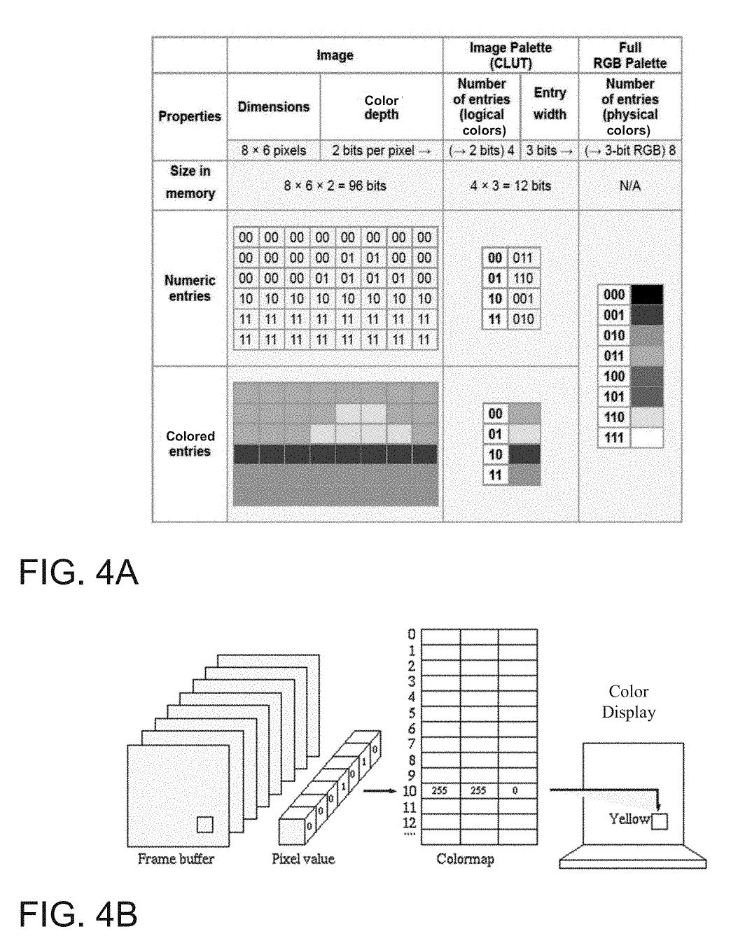

Human perception of both contrast and color saturation are closely related to luminance—the so-called Stevens and Hunt effects, respectively. So determining how to map a standard content color space onto an HDR color space for NPC is initially a subjective process, which may entail testing a plurality of different mappings with a plurality of different viewers to determine one or more mappings that consistently “look right”. The preferred mapping(s) can be stored in a color look-up table (CLUT), which is a mechanism used to transform a range of standard content input colors into a wider range of HDR colors. As noted, it can be embodied in a hardware device built into an imaging system, or a software block or function built into an image processing application executing on a tangible processor. In an embodiment, a color look-up table is used to convert the standard content logical color values stored in each pixel of video memory into higher bit depth HDR color values for display as NPC logical colors. In an embodiment, this conversion may be performed prior to applying a separate and distinct CLUT that converts the NPC logical colors into NPC physical colors, which may be represented as RGB triplets that can be displayed on an HDR display, for example. Alternatively, a single CLUT can be used that converts the standard content logical color values directly into higher bit depth NPC physical colors. In an embodiment, a color palette is implemented using a block of fast RAM which is addressed by a logical color and outputs red, green, and blue levels which drive the actual display. FIGS. 4A and 4B show abstract representations of exemplary CLUTs. FIG. 4A shows a 2-bit color palette being converted to a 3-bit color palette; in FIG. 4B an 8 bit logical color is converted to a physical color. Generating NPC physical colors from an 8 bit standard content color palette entails converting the increasing the color depth (e.g., from 8 bits to 10 bits, or 12 bits, or some other higher number of bits), and converting the increased color depth into physical colors appropriate for a particular HDR display, and replacing the brightest white and blue-white HDR values with the brightest possible value of physical color at about or near 489 nm wavelength.

NPC may be displayed not only on an electronic screen, but also on a projection surface, or a light field volumetric display system, etc., with the explicit purpose of stimulating increased energy levels and wakefulness via melanopsin stimulation for underexposed ipRGCs. Depending on certain characteristics of the NPC, this can mitigate light deprivation-induced conditions, affect changes in circadian rhythm, and can even stimulate an endogenous release of DMT (N,N-Dimethyltryptamine) within the brain.

In an exemplary aspect, a light source produces digital viewable images encoded to contain light at a specific wavelength or narrow range of wavelengths at about or near 479 nm. The images are displayed on a high-brightness capable display system, such as an HDR display of a digital device. Light at this wavelength or in this range is perceived visually as bright white, but is actually a light blue color similar to naturally occurring blue skylight. High intensity light of this color effectively stimulates the ipRGCs in humans at their peak responsiveness, which promotes stimulation of various biological mechanisms. The use of HDR video technology displays that are brighter than the screens most commonly used in prior art devices can enhance the effectiveness of this process, and can further benefit by using a novel method of encoding digital information to enhance its effectiveness.

Thus, enhanced melanopsin stimulation may be achieved by viewing digital content comprising pixels as high intensity colors at or near a target wavelength, such as very bright pixels at about 479 nm. Such digital content can be generated from existing original digital content using one or more of the following steps. If the original visual content is not digital, it can first be digitized in any convenient manner known in the art as the original digital content, and may then be further modified as will be described.

If the physical color space of the original digital content includes the target color (e.g., 479 nm), the intensity of pixels in the original content at or near the target color may be increased. However, if the bit representation of the content does not include an encoding for about 479 nm, the bit representation can be extended and modified to include such encoding. In an embodiment, applying the enhanced color space to standard content input to produce NPC output may include replacing the bit representations of a select narrow range of colors at or close to 479 nm, or any other wavelength that may be found to stimulate the MVS or some other human biological structure or element, such as the pineal gland or a virtual or latent parietal eye.

In an embodiment, the content's color space can be modified to more effectively stimulate the MVS by increasing the content's bit depth. For example, if an original image color bit depth of 8 bits cannot be made to represent a target color, converting the image to a higher bit depth (e.g., 10 bit, 12 bit, 14 bit, 16 bit, 32 bit, etc.) that can be made to represent colors at or near 479 nm wavelength. Moreover, by increasing the intensity (brightness) of a color generated at or new 479 nm, the color may appear white to a viewer due to its brightness. Alternatively or in addition, the light may be controlled using a feedback loop to produce a peak response in the ipRGC's. This can be done using one or more of the following exemplary aspects. A software module can be created which, when executed on a processor, generates a viewable environment using a HDR light source as a mobile VR or AR experience, or other computer software. Or, NPC can be generated as an original master version of computer generated imagery (CGI) or CGI-enhanced content; or regular standard content digital content may be converted to NPC via a previously described novel virtual effects (VFX) process to produce a new NPC master. Real time conversion of standard content digital content via the use of a 3D look up table (LUT) on appropriately capable display systems such as HDR computer displays, HDR TVs, HDR mobile devices, and HDR head mounted displays (HMDs).

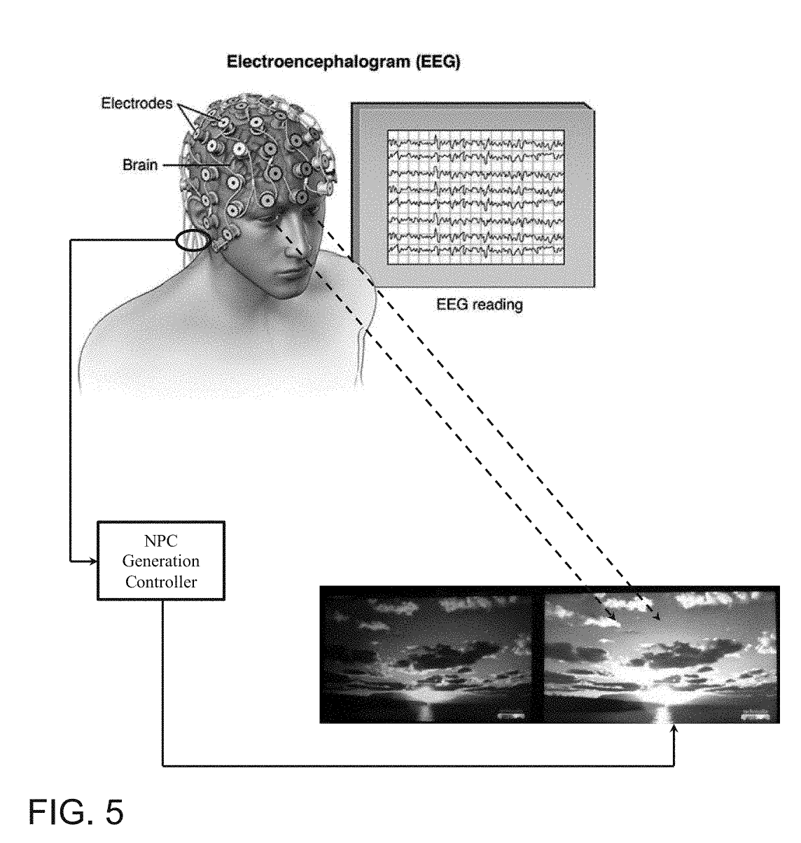

NPC may also employ nonrealistic dynamic depth augmentation, for example for stereoscopic NPC. This involves several layers of depth augmentation based on 3 separate Z-depth augmentations. Base layer natural depth map generation, a depth script augmentation which is a context-based animated augmentation, and a third underlying layer of depth augmentation via the use of mathematical form constants overlaid over distinct planar surfaces. The second and third layers of depth augmentation can be effected by a closed neurofeedback (NFB) loop system. NFB, also called neurotherapy or neurobiofeedback, is a type of biofeedback that uses real-time measurements of brain activity, such as electroencephalography (EEG). In NFB, low latency EEG sensors may be placed on the scalp to measure brain activity, and the measurements are used to modify NPC playing on an HDR video display, and/or binaural sound signals. FIG. 5 is an illustration of this type of system. As shown, electrodes are placed around the cranium of a subject viewer, and EEG signals are generated, as usual. However, the EEG signals are fed into an NPC generation controller. The controller uses the EEG signals to modify the NPC colors and/or intensity being displayed on an HDR TV and viewed by the subject. The controller keeps track of changes in the EEG signals and the state of the HDR TV, and correlates the two. Based on the correlation, the controller adapts the HDR TV output to promote the EEG profile desired. This can be done, for example, by modifying in real time the CLUT.

As noted, in an exemplary embodiment a novel method may be used to convert existing standard content digital content to Neurally Primed Content, such as for entertainment, wellness, and/or medical purposes. The standard content digital content is converted to a higher than 8 bit color depth. In an embodiment, a 32 bit float may be used in a complementary color space for HDR video (e.g., HDR-10, Dolby vision, Rec. 2020, Rec. 2100, or higher color space).

If NPC content is produced prior to viewing, such as by a compositor in a lab, any convenient method of matte extraction may be used for areas of the images having a predetermined wavelength or range of wavelengths. A luminance matte may be created and used as a guide to replace the existing matted bright color values, with a corresponding range of brighter lightness values, or with a select specific bright color value light enough to be perceived by a viewer as essentially similar. Once the matted area of the image has been identified and the specific replacement color wavelength or range selected, a user such as a compositor selects the magnitude of the lightness desired and remasters the content as NPC, i.e., as a new rendered file. Alternatively, for real time display conversion a lookup table (LUT) can be applied to the digital content in real time. In an embodiment, instead of replacing the original colors the LUT can be utilized by a capable display system as a non-destructive overlay to any piece of content provided.

Thus, through the use of a computer program for digital content creation (DCC) such as Maya or Nuke, an NPC creation artist can either create a new CG content file, or modify an existing digital imagery or video file to include higher lightness values in certain areas or objects in the scene. These files may then be output in a generic HDR compatible file format, or saved in a format specific to a particular viewing environment. For example, an NPC file may be produced for viewing on a specific smart phone with a known HDR screen type, and/or running a specific video player app, to allow for proper playback and display optimization of the generated NPC content on that device or player.

In an exemplary usage case, conversion of existing non-NPC digital images or video to NPC content may be performed by a content creator using a visual effects (VFX) application such as Nuke or After Effects to create a matte of the desired area, for example by luminance based matte extraction, rotoscopy, chroma keying or any other method of matte extraction.

After a matte has been created a color correction may be applied to the image or footage in color bit-depth higher than 8 bit to generate new color values with values higher than 1. These color values may not actually be white, but may be a specific pre-determined color value or range based on the corresponding desired color wavelength that has been determined to generate a peak ipRGC response. The methods of color correction or augmentation are varied and depend on the contextual parameters of both the content and its intended use. In some cases a straightforward color correction operation may be applied. In others a completely new element may be introduced via a composite operation. Regardless, by coding these new color values above 1, when the content is displayed via the use of HDR capable display systems they will be perceived as white light source but in actuality will output a different wavelength than the original white light wavelength.

The same process may also be applied when dealing with the color values of volumetric point data such as light fields, with the added benefit of much more granular control over the volumetric structure of the lighting effect, its frequency, and its falloff.

In a first illustrative usage case, a digital movie is presented on a HDR display with Neurally Primed Content generated on the fly by the display itself. The display measures all of the pixels with a brightness over a certain threshold, and replaces them with select appropriate super peak frequency skylight color values in those areas. Thereby, Neurally Primed Content may be created in real time based on non-NPC content provided.

In a second illustrative usage case, a computer generated (CG) NPC creation software package running on a computer workstation may offer tools to create graphic processing unit (GPU)-driven scenes, rendered images, and generated light fields coded with light values appropriate for NPC directly from the light source and shaders within the host application.

In a third illustrative usage case, a CG scene is produced of a beach scene comprising bright features such as a bright blue sky, the sun, and the white sand of the beach. These bright elements may be specifically encoded to include light with wavelength(s) at about 479 nm, which is the frequency providing peak stimulation of the melonospin vision system. The viewer, simply by staring into the image of the sun on an HDR capable display, stimulates their biological MVS to obtain corresponding medical and wellness benefits.

In a fourth illustrative usage case, a mobile VR app where users use their HDR-10 Capable mobile device with a mobile VR head-set solution when activated the app allows them to experience sustained targeted sequences of peak frequency skylight to treat medical conditions such as various sleeping disorders, and improve alertness when awake.

In a fifth illustrative usage case, a mobile device having an HDR-10 capable or similar display may be used to present non-NPC digital content modified to contain NPC appropriate light values included in, or overlaid on, the original content. The non-NPC content may be modified on the fly while being viewed. Alternatively, the non-NPC content may be modified before viewing. In either case, the non-NPC content may be converted into NPC content using a software application stored in a tangible memory and running on a tangible processor operatively coupled thereto. The software, memory and processor may reside in the viewing device, such as a mobile smart phone, smart TV, or the like. Or, they may reside elsewhere, such as in a server on a network operatively coupled to the viewing device, and streamed or stored on the viewing device before viewing.

In the same or a different embodiment, the viewing device may be used to view content that was originally generated to be viewed on an NPC capable device. Such content can be prepared to be viewed on a variety of specific mobile phone setups. In an embodiment, a standard framework may be established that each such viewing device conforms to. In this embodiment, the NPC content need not be modified to be viewed on different NPC-capable devices. In another embodiment, each viewing device may have a particular NPC setup including predetermined an NPC capable display and/or an NPC capable mobile app for viewing NPC content. In this embodiment, multiple versions of NPC content may be produced, each version generated for viewing on a particular setup.

Thus, MVS stimulation benefits may be obtained simply by viewing NPC versions of ordinary shows, movies, and the like. In addition to simply looking at a display device like an NPC capable TV, computer screen, or mobile phone, in an exemplary embodiment a user may wear an NPC enabled head mounted display (HMD) operatively coupled to a mobile device they are carrying. This arrangement can constitute a VR or AR HMD for the duration of the viewing experience. NPC can be used with any NPC capable mobile VR headset solution, such as appropriately modified Google Cardboard or Daydream, Samsung VR, and the like.

Moreover, a user may choose to use any NPC capable viewing environment to experience a therapeutic treatment for a medically diagnosed MVS-related condition, such as seasonal affective disorder (SAD). Such a treatment may last, for example, from about five minutes to about 30 minutes or more. During treatment, the user may be seated, lying down, or in some other meditative pose. In an embodiment, a pre-recorded therapeutic program may be viewed containing actual or CG images and/or video designed to induce a relaxing, meditative state.

In an embodiment, NPC visual content may be arranged to be played in conjunction with specially produced audio using headphones, earbuds, or the like, that provide distinct, isolated audio to each ear, such as “binaural beat” audio. Such audio provides a flat, even tone to each ear individually, but at slightly different tones or frequencies. This arrangement surprisingly can result in subjectively “hearing” a vibrato. This vibrato sounds similar to the interference effect that would be caused by two speakers in close proximity, each speaker playing a different tone, with the resulting interference pattern heard by both ears. However, in the case of headphones, each ear can hear only a single flat even tone, so there can be no physical interference causing the perceived vibrato. Instead, the brain itself creates the vibrato, or “beat”, which requires a mental process involving both hemispheres of the brain working in cooperation. Such binaural beat audio can be generated on the fly using an application stored in memory and running on a processor, or it can be pre-recorded. In addition, the binaural beat audio can be provided alone by itself, or it can be combined with ordinary audio recordings such as relaxing music, or a verbal audio track such as guided meditation.

This type of audio can produce enhanced feelings of calm and relaxation in a user. In an embodiment, a therapeutic treatment recording can be produced having both an NPC visual component and a binaural beat audio component, designed to transport the user to a surreal natural environment meant to calm them down and induce them to fall into a profound meditative state. With the use of basic color cognitive science, binaural audio, and a voice over, the user can be guided into a meditative state in which they may close their eyes. Even with eyes closed, the NPC vision-based stimulation of the melanopsin vision system can be done safely and effectively.

In an embodiment of an NPC-based therapeutic VR session designed to treat jet lag, the user may begin the session with their eyes closed, and may keep them closed for the entire session. A light source within the VR environment may become brighter and fill the entire viewable field. The wavelength (color) of the light source may be modified over a predetermined range as the user's ipRGC response is measured to identify the wavelength that produces the peak response. The ipRGC response can be tracked in any manner known in the art, and can then be used in a feedback loop to cause the light source to produce the specific wavelength producing the peak response. Thereby, the user's circadian rhythm may be beneficially influenced by stimulating the MVS, thereby promoting a state of wakefulness and well-being.

While the subject disclosure has been described with reference to exemplary embodiments, it will be appreciated by those skilled in the art that various changes may be made and equivalents may be substituted for elements thereof without departing from the scope of the subject disclosure. In addition, modifications may be made to adapt a particular situation or material to the teachings of the exemplary embodiments without departing from the essential scope thereof. It is to be understood, therefore, that the exemplary embodiments not be limited to the particular aspects disclosed, but it is intended to cover modifications within the spirit and scope of the subject disclosure as discussed above.

I claim:

1. A system for stimulation of a Melanopsin vision system of a user comprising:
Melanopsin stimulant content;
means for converting standard content to Melanopsin stimulant content; and
an apparatus configured to deliver the Melanopsin stimulant content to a user.

2. The system of claim 1, wherein the apparatus configured to deliver the Melanopsin stimulant content comprises an HDR enabled display, an array of at least one light emitting diode, or a head mounted display system.

3. The system of claim 1, wherein the Melanopsin stimulant content includes digital content encoded with specific visible and nonvisible frequencies of light.

4. A method for creating high dynamic range (HDR) content by converting standard content comprising:
analyzing a color and brightness content of a piece of standard content;
based on the analysis, developing a color lookup table (CLUT) for use in converting the standard content color and brightness content to a higher bit depth representation of the color and brightness content;
using the CLUT and the standard content to create a high dynamic range (HDR) logical representation of the content having the higher bit depth; and
creating a physical color representation of the higher bit depth HDR logical representation of the content for viewing on an HDR display.

5. The method of claim 4, wherein the HDR logical representation of the content having the higher bit depth comprises Melanopsin stimulant content having a HDR logical representation of the content having the higher bit depth.

6. The method of claim 5, wherein the step of creating the HDR logical representation of the content comprises encoding alternative specific light frequencies in visible light values of at least one generated digital representation, including an image, video, or plenoptic data set operable to stimulate intrinsically photosensitive retinal ganglion cells (IPRGC) and a Melanopsin vision system of a user to generate one or more biological responses.

7. The method of claim 4, wherein the physical color representation comprises a Melanopsin stimulant content physical color representation of the higher bit depth HDR logical representation of the content.

8. The method of claim 4, wherein the physical color representation comprises a Neurally Primed Content (NPC) physical color representation of the higher bit depth HDR logical representation of the content.

9. The method of claim 4, wherein the step of creating the HDR logical representation of the content is accomplished on a server computer in communication via a network to a user computing device having an HDR display.

10. The method of claim 4, wherein the step of creating the physical color representation of the HDR logical representation of the content is accomplished on a server computer in communication via a network to a user computing device having an HDR display.

11. The method of claim 4, wherein the creation of the HDR logical representation of the content is accomplished on a user computing device having an HDR display.

12. The method of claim 4, wherein the creation of the physical color representation of the HDR logical representation of the content is accomplished on a user computing device having an HDR display.

13. A system for creating high dynamic range (HDR) content based on standard content for viewing on an HDR display comprising:
a standard content analysis component;
a lookup table component;
an HDR logical content creation component; and
a physical color creation component.

14. The system of claim 13, wherein the HDR logical content creation component is configured to create HDR Melanopsin stimulant content.

15. The system of claim 14, wherein the physical color creation component is configured to create a Melanopsin stimulant content physical color representation of the HDR Melanopsin stimulant content.

16. The system of claim 13, wherein the HDR logical content creation component is configured to create HDR Neurally Primed Content (NPC).

17. The system of claim 16, wherein the physical color creation component is configured to create an NPC physical color representation of the HDR Neurally Primed Content.
